(19) United States
(12) Patent Application Publication
(10) Pub. No.: US 2005/0064421 A1
(43) Pub. Date: Mar. 24, 2005

(54) Profiling of the immune gene repertoire

(75) Inventors: Mathias Gehrmann, Leverkusen (DE); Stephen Schwers, Koln (DE); Marcus Weidler, Langenfeld (DE)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Healthcare AG

(21) Appl. No.: 10/496,260

(22) PCT Filed: Nov. 15, 2002
(86) PCT No.: PCT/EP02/12822

(30) Foreign Application Priority Data
Nov. 23, 2001 (GB) 0128153.4

(51) Int. Cl. C12Q 1/68

(52) U.S. Cl. 435/6

(57) ABSTRACT

The present invention relates to a method for the profiling of the antibody and T-cell receptor mRNA repertoire of an organism. Profiles that are generated describe the current immune status. This knowledge is useful for the diagnosis and prediction of disorders and for the identification of therapeutic drugs and proteins.

### FIELD OF THE INVENTION

The present invention relates to a method for the profiling of the antibody and T-cell receptor mRNA repertoire of an organism. Profiles that are generated describe the current immune status. This knowledge is useful for the diagnosis and prediction of disorders and for the identification of therapeutic drugs and proteins.

### BACKGROUND OF THE INVENTION

The adaptive immune system of vertebrates allows a specific response to a huge variety of antigens and pathogens. This response is based on the existence of B and T lymphocytes, that exert their function through B cell receptors (BCR) or T cell receptors (TCR) which are assembled through somatic gene recombination during B and T cell development. BCR and TCR are bound to the membrane of B and T lymphocytes together with coreceptors, that mediate specific signals after recognition of ligands. In addition B lymphocytes can secrete BCR in form of specific antibodies, that are also able to exert immune reactions.

Due to the recombination process (that is known as V(D)J recombination) a high variability of BCR and TCR is possible, that is orders of magnitude higher than the number of lymphocytes in the vertebrate. E.g., while an estimated number of 10¹⁴-10¹⁵ different BCR specificities can theoretically be created by a B cell, only 10¹¹ B cells exist in the human.

B-Cells

B lymphocytes are the primary mediators of humoral immunity by production of antibodies, that are able to specifically bind to foreign invaders like viruses, parasites, and bacteria and initiate their destruction. Antibodies are globular proteins that circulate in blood, lymphatic, and bodily fluid. The humoral immune response is based on the recognition of antigen by the antibody. In the destruction of antigen the antibody fulfills three main functions: (1) The activation of the major effector of the humoral branch, the complement system, a system based on various proteins, (2) the binding to antigens, thus eliminating their capability to harm, and (3) the recognition by Fc receptors on professional phagocytic cells. The antigen is first recognised by membrane attached antibodies (IgM and IgD) on the surface of a specific B cell. It is then endocytosed and presented on a class II MHC activating T-helper 2 cell. Other antigen presenting cells like dentritic cells or macrophages can also activate T-helper 2 cells. Afterwards, they will attach and stimulate B-cells by releasing cytokines such as IL-4 to initiate development into plasma cells. These plasma cells produce and secrete significant quantities of secreted antibodies, which bind to antigen either free in solution or on the surface of a foreign cell, forming a precipitate and causing a conformational change in the antibody Fc segment. This change allows the complement system to initiate lysis of the foreign cell in a cascade that begins with the binding of the antibody to a microbial surface antigen. Otherwise, if the antigen is not membrane-attached but precipitated by antibodies, “Innocent-Bystander Lysis” occurs, killing a vital cell. In addition, cleavage products of proteins of the complement system serve as opsonins, which are responsible for calling in neutrophils and macrophages. This initiates further sensitisation of the immune system against the foreign antigen and an inflammatory response. Along with the function of antibodies as complement system activator (especially IgM), they also serve as opsonins themselves, calling in neutrophils.

As the efficiency of a vertebrate to mount a humoral immune response is dependent on the existence of specific antibodies, the complete collection of expressed immuno-globulins (i.e., the immunoglobulin ‘repertoire’) is a determinant of the organisms immune status.

However as V(D)J recombination happens through somatic rearrangements of distinct genomic loci in B and T cells, also the collection of genomic V(D)J rearrangements of a vertebrate can be called a ‘repertoire’. Although some of those rearrangements may not be actually expressed (as they're nonproductively rearranged) knowledge of the genomic rearrangement status of B and T cells allows an extrapolation of the expressed immunoglobulin and T cell receptor repertoire.

T-Cells

T lymphocytes are the primary mediators of cellular immunity in humans, occupying an essential role in immune responses to infectious agents (e.g., viruses and bacteria) and in the body's natural defenses against neoplastic diseases. Likewise, T lymphocytes play a central role in acute graft versus host disease, wherein the immune system of a host attacks (rejects) implanted tissue from a foreign host, in autoimmune disorders, in hypersensitivity, in degenerative nervous system diseases, and many other conditions. A T cell immune response is characterised by one (or more) particular T cell(s) recognizing a particular antigen, secreting growth-promoting cytokines, and undergoing a monoclonal (or oligoclonal) expansion to provide additional T cells to recognise and eliminate the foreign antigen.

Each T cell and its progeny are unique by virtue of a structurally unique T cell receptor (TCR), which recognises a complimentary, structurally unique antigen. In general, T cells produce either of two types of TCR. The γδ receptor is found on <5% of T lymphocytes. It is synthesised only at an early stage of T-cell development. TCR αβ is found on >95% of lymphocytes. It is synthesised later in T-cell development than γδ. The TCR αβ is responsible for helper T cell function in cell-mediated immunity and for killer T cell function in cell-mediated immunity. TCRs recognise a peptide in a groove on the surface of a MHC protein. The result of this specific interaction is signaling through the CD3 complex. Depending upon the stage of differentiation of the T cell and on the co-stimulatory signal, this can lead to T cell proliferation, to T cell effector function, to T cell anergy or to cell death.

The structure and basis of the diversity of the TCR is now well known. Diversity is generated through somatic recombination at the TCR loci. This recombination involves three different segment types: V (variable), D (diversity) and J (joining) segments, resembling recombination in immunoglobulins. Additional diversity is generated at the junction of the segments during the recombination process. The organisation of TCRα resembles that of Ig κ, with V genes separated from a cluster of J segments that precedes a single C gene. In addition to the α segments, this locus also contains δ segments. The organisation of TCR β is different, with V genes separated from two clusters each containing a D segment, several J segments, and a C gene. Within the T cell receptor α and β chain variable regions are hypervariable regions similar to those found in immunoglobulins, where they form the principal points of contact with antigen and thus are referred to as CDR (complementarity determining regions). Based on the analogy with immunoglobulins, these TCR hypervariable regions are thought to loop out from connecting β-sheet TCR frame-work sequences. Two CDRs (CDR1 and CDR2) are postulated to contact pre-dominantly major histocompatibility complex (MHC) peptide sequences, whereas a third, centrally-located CDR (CDR3) is believed to contact peptide bound in the MHC antigen binding groove.

For TCR αβ cells, the repertoire available in the periphery is not only the result of the random processes of recombination. Central repertoire shaping occurs during T cell development in the thymus, both by positive selection of T cells with the potential for recognising autologous MHC molecules, and by the destruction of overtly self reacting T cells (negative selection).

The characterisation of T cell responses in normal physiological and pathological situations, including auto-immunity, response to infectious agents, alloimmunity, and tumor immunity, is a key to understand disease control by the immune system and is beginning to play an important role in many clinical situations.

The totality of BCRs and TCRs being expressed by a vertebrate at a certain point in time, i.e., the vertebrates immune gene repertoire, mirrors the vertebrate's immune status. Hence, from a concise analysis of an vertebrate's immune gene repertoire, one can draw conclusions on the immune status and on the susceptibility to diseases. In addition, ongoing diseases and inflammatory reactions can be assessed via the immune gene repertoire, and decisions for treatment may be concluded.

Another level of complexity is added to the immune system by signaling molecules like chemokines, cytokines and membrane bound signaling molecules. Those immune modulators allow a crosstalk between T, B and other cells of the immune system. Hence an expression analysis of those signaling molecules does also allow an assessment of the organisms immune status, complementing the information obtained by a BCR and TCR repertoire analysis.

State of the Art

Various immunoglobulin (Ig) repertoire analyses have been performed in the past, and have shown that a change in the Ig repertoire can be related to different physiological stages of the organism. More specifically it was found, that diseases like sarcoidosis, hepatitis, multiple sclerosis, lymphomas and graft versus host disease are associated with a shift in the Ig repertoire.

However all previous repertoire analyses were hampered by their experimental design, which did not allow for high throughput analysis. Previous analyses were performed using colony hybridisation to filters, sequencing or complementarity determining region (CDR) spectratyping. Those techniques were very laborious and did not allow to assess and compare the Ig and TCR repertoire of a statistically significant number of individuals.

One example of a Ig repertoire analysis was provided by Williamson et al., Proc Natl Acad Sci USA., 13; 98(4): 1793-8, 2001, who extracted RNA from acute plaques of multiple sclerosis patients. cDNA was prepared and antibody heavy and light chains were PCR amplified and subcloned. However only selected Ig chains were subsequently analysed. For this, single light and heavy chains were transfected into an eukaryotic cell line and recombinant whole Ig molecules were expressed. The specificity of the resultant complete Ig molecules was studied by immuno-cytochemistry and FACS analyses. Williamson et al. (2001) made use of a labour-intensive procedure, that did by no means cover the complete Ig repertoire. Probably due to the complexity of the experimental system no healthy control individual was included. However Williamson et al. (2001) were able to show, that autoimmune Ig repertoires exist in MS patients.

Another example is provided by Baxendale et al., Eur J. Immunol., 30(4): 1214-23, 2000, who established B cell hybridomas from human individuals. The Ig repertoire of those individuals was subsequently characterised by ELISA and sequencing. Using this procedure Baxendale et al. (2000) were able to analyse human immune responses to S. pneumoniae and various S. pneumoniae vaccines.

Intensive investigative efforts have been directed to developing improved methods for monitoring the T cell repertoire to better understand, monitor, an modulate the immune system. Methods of T-cell repertoire analysis include random sequencing, RNase protection assays (Okada et al., J. Exp. Med. 169: 1703-1719, 1989; Singer et al., EMBO J. 9: 3641-3648, 1990), TCR mini-libraries in E. coli generated by anchored or inverse PCR (Rieux-Laucat et al., Eur. J. Immunol. 23: 928-934; Uematsu et al., Immunogenetics 34: 174-178, 1991), and V-gene usage analysis using specific monoclonal antibodies (mabs) when available (Genevee et al., Int. Immunol. 6: 1497-1504, 1994). Many of the more successful advances in T cell repertoire analysis have involved polymerase chain reaction (PCR) methodologies directed to measuring T cell receptor repertoires. See generally Cottrez et al., J. Immunol. Methods, 172: 85-94, 1994.

Oaks et al. (Am. J. Med. Sci., 309(1): 26-34, 1995) reported a PCR-based method of T cell repertoire analysis comprising extracting RNA from a cell sample, synthesizing cDNA from the RNA, and amplifying aliquots of the cDNA via PCR (around 40 cycles) using family-specific Vα and Vβ oligonucleotide primers. The PCR products were analyzed by electrophoresis on a 2% agarose gel followed by Southern blotting using α-chain or β-chain constant region gene probes, wherein expression of a specific TCR Vα or Vβ family was considered positive if a distinct band was detected. The method was useful for distinguishing tissue rejection lesions versus non-rejection lesions in cardiac allograft patients. However, the Southern blot analysis provides suboptimal information about the T cell repertoire within a particular Vα or Vβ gene family. For reasons, see also Dietrich et al. (Blood, 80(9): 2419-24, 1992).

In European Patent Application No. 0653 493 A1, filed 30 Apr. 1993, the inventors reported a PCR-based method of T cell repertoire analysis comprising extracting RNA from a cell sample, synthesising cDNA from the RNA, and amplifying aliquots of the cDNA via PCR using family-specific Vβ oligonucleotide primers. The PCR products were then analyzed using a “single strand conformation polymorphism” (SSCP) technique wherein the PCR-amplified cDNA is separated into single strands and electrophoresed on a non-denaturing polyacrylamide gel, whereby DNA fragments having the same length are made further separable by differences in “higher order structure.” Using this method, the amplified DNA from peripheral blood lymphocytes reportedly is observed generally as a “smear” whereas the detection of a single band amidst a smear is indicative of T cell clonal expansion.

Cottrez et al., reported a PCR-based method of T cell repertoire analysis comprising extracting RNA from a cell sample, synthesising cDNA from the RNA using oligo-dT primers, and amplifying aliquots of the cDNA via PCR (around 25 cycles) using family-specific Vβ oligonucleotide primers. The PCR products were analyzed on a DNA sequencer and reportedly contained 6-11 discrete fragment peaks spaced by 3 base pairs in length, representing “all” various sizes of the CDR3 region. See also Gorski et al., J. Immunol., 152: 5109-5119 (1994).

Puisieux et al., J. Immunol., 143 2807-18 (1994), reported a PCR-based method of T cell repertoire analysis comprising determining VDJ junction size patterns in twenty-four human TCR Vβ subfamilies. The TCR Vα subfamilies were not characterised. These investigators employed the method to analyze T cells infiltrating sequential malignant melanoma biopsies for the presence of clonal expansions, and detected such expansions over a more or less complex polyclonal background. Their study highlights the utility of T cell repertoire analysis methods for monitoring neoplastic conditions and treatments for such conditions.

The method of T cell repertoire analysis of Puisieux et al. reportedly includes the steps of extracting RNA from cells, synthesizing cDNA from the RNA using oligo-(dT) primers, and amplifying aliquots of the cDNA via PCR using family-specific Vβ oligonucleotide primers. Potential clonal expansions in the PCR products were tentatively identified in families where a single fluorescence peak (on a sequencing gel) corresponded to 40% of the total fluorescence intensity of all of the peaks in the family. To “refine” the T cell repertoire analysis, a second set of Vβ family-specific PCR reactions of interest were further subjected to primer extension “run off” reactions using a fluorophore labelled Cβ primer and/or using thirteen Jβ-family-specific, fluorophore-labelled Jβ primers. The run-off reaction products were then analyzed on additional sequencing gels.

The same investigative group has more recently elaborated on their T cell repertoire analysis methods. See Pannetier et al., Immunol. Today, 16: 176-181, 1995. The group reports that the Vβ families are easier to analyze by PCR than Vα families. Nonetheless, their Vβ analysis methods involve twenty-five Vβ family-specific PCR amplifications (each of which yields an average of eight peaks), twenty-five Cβ “run-off” reactions, and 325 Jβ “run-off” reactions (25 Vβ×13 Jβ=325). Each “run-off” reaction is analyzed by electrophoresing an aliquot on a polyacrylamide gel.

In patent application WO 97/18330 Dau et al. Claim a novel method of analyzing the T cell repertoire which they call intrafamily fragment analysis of the T cell receptor CDR3 region and which is distinguished from what they call interfamily analysis. During interfamily analysis the PCR products form each family are quantitatively compared, but it is practically impossible to optimise primer efficiencies and to stop all reactions in log phase for all V beta families. Therefore, judgements about the relative amounts of TCR gene expression between families can be unreliable. In intrafamily analysis fragments generated by a single V beta primer are compared, thereby avoiding the optimisation of reaction conditions necessary for interfamily analysis.

Using any of the known methods above to characterise the immune gene repertoire of a vertebrate allows only to discriminate between different TCR or BCR family members upon the criterion of the specificity of the primers applied, and of the length of the amplified PCR products. These analyses are very tedious to perform and still, the information content of the results obtained is rather low. Furthermore, none of the above mentioned methods to profile the immune gene repertoire allows for a high throughput analysis, nor does it provide for a comprehensive description of the T-cell and/or B-cell repertoire of a vertebrate animal.

### SUMMARY OF THE INVENTION

The invention provides methods for the high throughput profiling of a vertebrate's immune gene profile. In these methods sequences containing at least part of the variable regions of antibody and/or T-cell receptor genes are isolated and/or amplified from DNA, total RNA or mRNA isolated from B- or T-cells. Amplification is done using suitable oligonucleotides that are specific for the gene segments coding for the variable and/or constant region of antibody genes and/or the variable and/or constant region of T-cell receptor genes. The pool of amplified nucleic acids from variable regions is analysed by hybridizing the amplification products on an oligonucleotide array. The hybridised molecules on the oligo-nucleotide array are detected by appropriate methods known in the art and the hybridisation pattern is correlated with the immune status, e.g., previous or current diseases, protection against future diseases, or prediction of disease progression. Patterns that correlate to protection against a disease or disease progression can be used to identify the responsible antibodies or T-cell receptor genes. Once a particular antibody or T cell receptor has been identified it is also possible to identify the antigen or pathogen the antibody or T cell receptor is specific for.

### BRIEF DESCRIPTION OF THE DRAWINGS

FIG. 1 shows the TCRB 3′ primer consensus sequence (SEQ ID NO:1)

FIG. 2 shows the sequence of TCR C BETA1 (SEQ ID NO:2)

FIG. 3 shows the sequence of TCR C BETA2 (SEQ ID NO:3)

FIG. 4 shows the primer T7(CH1) (SEQ ID NO:4)

FIG. 5 shows the oligo V beta1 (SEQ ID NO:5)

FIG. 6 shows the oligo V beta2 (SEQ ID NO:6)

FIG. 7 shows the oligq V beta3 (SEQ ID NO:7)

FIG. 8 shows the oligo T7-C-beta (SEQ ID NO:8)

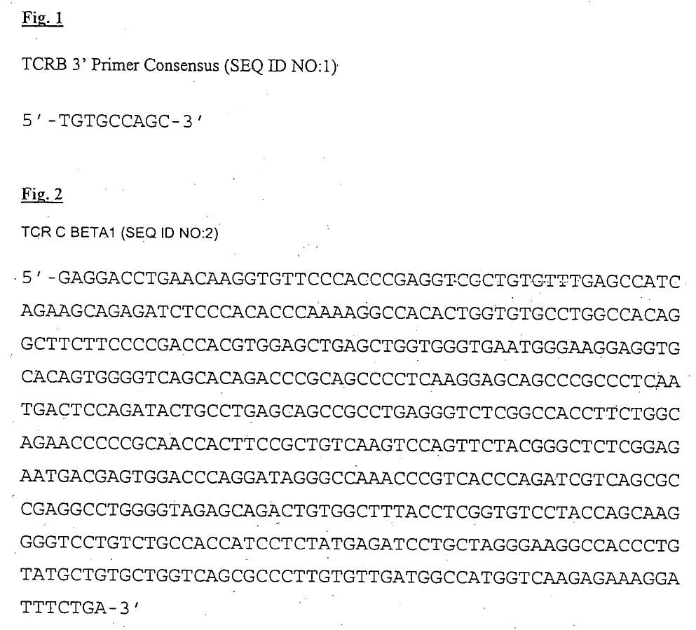

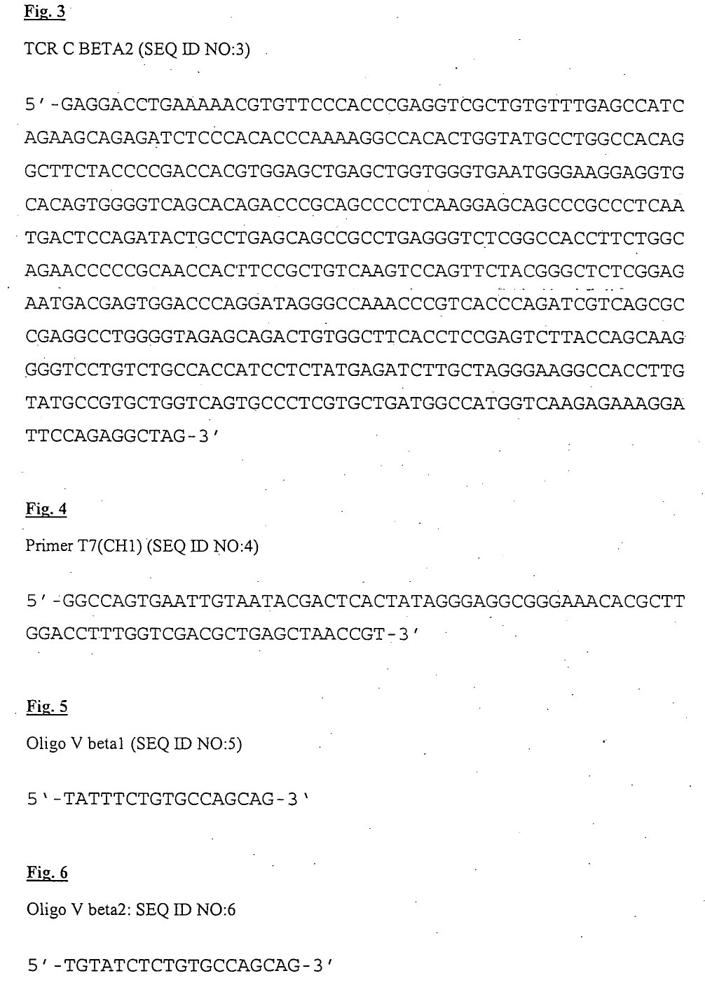

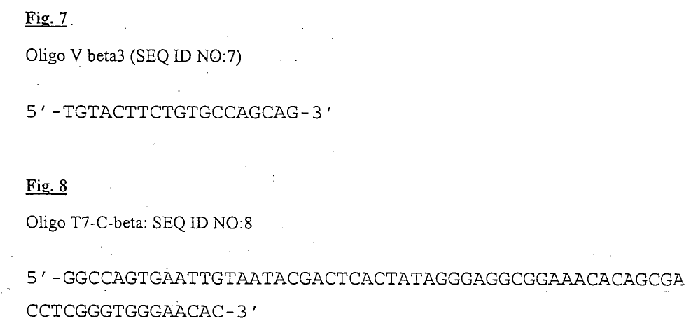

### DETAILED DESCRIPTION OF THE INVENTION

A method has been developed that allows the profiling of rearranged antibody and T-cell receptor genes of a vertebrate. First, cells are obtained from a Vertebrate. These cells may be derived from any source, as long as the cell sample contains T- or B-lymphocytes. Peripheral blood is a preferred source for obtaining cells from the vertebrate. Also preferred are cells that are derived from a body fluid of the vertebrate, the fluid being selected from a group of fluids consisting of synovial fluid, cerebrospinal fluid, lymph, bronchioalveolar lavage fluid, gastrointestinal secretions, saliva, urine, and tears. In another preferred embodiment, the cells are derived from a tissue of the individual, e.g., by performing a tissue biopsy. When assaying for a particular disease condition the selection of appropriate cell sources will be apparent to those of ordinary skill. For example, to assay for autoimmnune disorders affecting the joints (e.g., rheumatoid arthritis), synovial fluid is a preferred fluid from which to derive cells. To assay for disorders affecting the liver (e.g., hepatitis, primary biliary cirrhosis), the liver is a preferred tissue from which to derive cells.

T- or B-cells may be extracted from the cell sample by fluorescence activated cell sorting (FACS), magnetic cell sorting (MACS), leucapheresis, density gradient centrifugation or other suitable techniques. Under some circumstances it may be advantageous to further subdivide the isolated T- or B cell populations into functionally distinct subsets using FACS, MACS or other appropriate techniques. Those functionally distinct subsets may enclose different developmental stages of lymphocytes as identified by cell surface molecules or other markers that allow their distinction.

DNA, total RNA or mRNA is prepared from the obtained cell population and used as a template for the specific amplification of sequences contained at least in part in the variable region of immune genes. One possible amplification method is the generation of immune gene antisense RNA (aRNA) by in vitro transcription. The first step in this amplification method involves synthesising an immune gene specific primer that is extended at the 5′ end with an RNA polymerase promoter such as the T7 or SP6 promoter. This oligonucleotide can be used to prime mRNA populations for immune gene specific cDNA synthesis. Specificity is conferred by the 3′ part of the oligonucleotide which is complementary to a sequence within the immune genes. This sequence is shared in at least a subfamily of antibody or T-cell receptor genes. In one embodiment of the invention, the sequence is complementary to a sequence in the CH1 region of antibody heavy chains belonging to the IgG, IgM, IgA, IgD and/or IgE class. In another embodiment of the invention, the sequence is complementary to a sequence in the constant domains of TCR alpha, TCR beta, TCR gamma or TCR delta. After the first strand cDNA is synthesised, the second strand cDNA is made, followed by RNA nuclease treatment to degrade the RNA and treatment with T4 DNA polymerase to generate a double-stranded molecule. This double-stranded cDNA can then be used for amplification by utilising the incorporated RNA polymerase promoter to direct the synthesis of aRNA.

An alternative amplification method is the polymerase chain reaction. The primers used for amplification of variable regions of immune genes are complementary to sequences that allow at least the amplification of an immune gene subfamily. In one embodiment of the invention the CDR3 region of heavy or light chains of immunoglobulins or T cell receptors are amplified using primer located 5′ and 3′ of the CDR3. Amplification of rearranged human immunglobulin genes can be performed using oligonucleotides as described in Sblattero and Bradbury, Immuno-technology 3(4): 271-8, 1998; or Wang and Stollar, J Immunol Methods, 244(1-2): 217-25, 2000. The primers described in those references were shown to assess the majority of human immunoglobulin genes. Amplification of human immunoglobulin CDR3 regions can be done as described in Efremov et al., 1995.

In another embodiement of the invention the immune genes are amplified using primers located 5′ and 3′ of the respective V(D)J regions known in the art (Küppers et al., EMBO J. 1993 Dec. 15; 12(13): 4955-67; Roers et al., Am J Pathol. 2000 March; 156(3): 1067-71; Willenbrock et al., Am J Pathol. 2001 May; 158(5): 1851-7; Muschen et al., Lab Invest 2001 March; 81(3): 289-95).

In yet another embodiement of the invention the expressed immune genes are reversely transcribed using an oligo dT primer or an immune gene specific primer complementary at least in part to sequence in the immune gene constant region and subsequently amplified by PCR using appropriate primers. Primer recognition sites may have been added prior to PCR by 1) attachment of a linker to the ends of the cDNA or 2) by tailing of the cDNA with unique nucleotide residues using the enzyme terminal desoxynucleotidyl transferase.

The amplified immune genes are the target molecules that need to be analysed by an oligonucleotide array, SAGE or related techniques. Target molecules may be labelled for subsequent detection on an oligonucleotide array. Labelling of amplified target molecules can be done according to methods known to those of skill in the art. One possibility is the incorporation of labelled nucleotides like biotinylated UTP or CTP during the in vitro transcription reaction. Another possibility is the labelling of target molecules after the amplification reaction e.g., by enzymatically modifying the 5′ end of the amplified nucleic acids by T4 polynucleotide kinase with γ-S-ATP and subsequent conjugation with biotin.

Labelled target molecules are then hybridised to an oligonucleotide array. The array consists of a variety of different oligonucleotides that have been immobilised or synthesised at specific locations on the array by methods known to those of skill in the art. Custom designed oligonucleotide arrays can be purchased (Affymetrix, Santa Clara, USA, Agilent Technologies, Palo Alto, USA).

In a preferred embodiment of the invention the oligonucleotide array consists of all possible oligonucleotide 8 mers, 9 mers, or 10 mers synthesised in situ or immobilised at known locations on the array. Another example of such an oligonucleotide array contains oligonucleotides designed to be complementary to a particular subset of immune genes. The sequence information for these oligonucleotides may be obtained by sequencing of cloned TCR or BCR receptor genes. Hybridisation and visualisation of hybridised target molecules is done according to methods known to those of skill in the art. In one embodiement, biotinylated target molecules not specifically bound to the array are washed away and specifically bound molecules may be stained using a streptavidin-phycoerythrin conjugate. After washing away unbound conjugate molecules the stained array may be scanned. The pattern of detected hybrisation complexes can be analysed and correlations between pattern and diseases can be identified.

Since the TCR is MHC restricted it is preferable to stratify patterns obtained from analysis of TCRs according to the genetic MHC background. The MHC genes can be determined by conventional methods like serum analysis with antibodies, PCR analysis using appropriate primer or by DNA array analysis using appropriate oligonucleotide probes.

Identification of a specific pattern of immunoglobulin- or TCR-transcripts that is associated with a disease may be employed to diagnose and to monitor the disease. Furthermore, identification of particular disease relevant sequences from immuno-globulins or TCRs may allow the isolation of the complete molecules and provide a basis for therapy. The therapies may involve ablation of immune cells carrying the particular immune gene, administration of compounds which inhibit binding of the immune genes to their target molecules or the expansion of cell carrying the desired immune genes.

An “immune gene”, within the meaning of the invention is a nucleic acid molecule coding for the amino acid sequence of an immune receptor, or fragments of said immune receptor.

An “immune receptor”, within the meaning of the invention shall be understood as being a molecule, or fragments of said molecule, which is involved in the immune response by detecting or binding to antigens or fragments of said antigens.

An “immune cell” within the meaning of the invention shall be understood as a cell which is involved in the immune response of a vertebrate, e.g., by expressing or carrying immune receptors which detect or bind to antigens. Immune cells can be, e.g., T-cells and B-cells.

The “immune gene repertoire” of a vertebrate animal is to be understood as being the totality of immune genes which is present in a vertebrate's body. A characterisation of the immune gene repertoire comprises, but is not limited to, the detection and/or the quantification of all or part of the immune genes present in a vertebrate animal.

For a sample of nucleic acid molecules to “represent the immune gene repertoire” of cells, the sample of nucleic acid molecules shall be understood as being a sample of nucleic acid molecules in which the distribution of immune genes resembles, or in a preferred embodiment is approximately similar to, the distribution of immune genes in said cells.

The term “immune disorder”, within the meaning of the invention, refers to a disease or another physical condition involving immune cells. Such disorders include but are not limited to autoimmune diseases, neoplastic diseases, infectious diseases, hyper-sensitivity, transplantation, and graft-versus-host disease, and degenerative diseases. Autoimmune diseases include but are not limited to rheumatoid arthritis, type I diabetes, juvenile rheumatoid arthritis, multiple sclerosis, thyroiditis, myasthenia gravis, systemic lupus erythematosus, polymyositis, Sjogren's syndrome, Grave's disease, Addison's disease, Goodpasture's syndrome, scleroderma, dermatomyositis, pernicious anemia, autoimmune atrophic gastritis, primary biliary cirrhosis, and autoimmune hemolytic anemia. Neoplastic diseases include but are not limited to lymphoproliferative diseases such as leukemias, lymphomas, Non-Hodgkin's lymphoma, and Hodgkin's lymphoma, and cancers such as cancer of the breast, colon, lung, liver, pancreas, skin, etc. Infectious diseases include but are not limited to viral infections caused by viruses such as HIV, HSV, EBV, CMV, Influenza, Hepatitis A, B, or C; fungal infections such as those caused by the yeast genus Candida; parasitic infections such as those caused by schistosomes, filaria, nematodes, trichinosis or protozoa such as trypanosomes causing sleeping sickness, plasmodium causing malaria or leishmania causing leishmaniasis; and bacterial infections such as those caused by mycobacterium, corynebacterium, or staphylococcus. Hypersensitivity diseases include but are not limited to Type I hypersensitivities such as contact with allergens that lead to allergies, Type II hypersensitivities such as those present in Goodpastures's syndrome, myasthenia gravis, and autoimmune hemolytic anemia, and Type IV hypersensitivities such as those manifested in leprosy, tuberculosis, sarcoidosis and schistosomiasis. Degenerative disease include but are not limited to Parkinson's disease, Alzheimer's disease, and atherosclerosis. “Suitable cells” within the meaning of the invention, shall be understood as being any group of cells containing B-cells and/or T-cells.

“Oligonucleotides”, within the meaning of the invention, are nucleic acid molecules of 5 to 100 nucleotides in length.

A “variable region of an immune gene” within the meaning of the invention, shall be understood as being the part of an immune gene which codes for the specific binding domain of an immune receptor. Examples for the variable regions of an immune gene are the regions of an immune gene coding for the CDR1, CDR2, and CDR3 regions of an immune receptor. Specific, within the meaning of the invention, does not necessarily mean absolutely specific.

“In vivo transcription”, within the meaning of the invention shall be understood as an experimental technique for the amplification of nucleic acid molecules as described by Phillips and Eberwine, Methods 1996 December; 10(3): 283-8.

“Conditions compatible with PCR”, within the meaning of the invention, shall be understood as conditions suitable for the annealing step of a PCR reaction. These conditions are well known to those skilled in the art. One example is given, e.g., in Sambrook et al., MOLECULAR CLONING: A LABORATORY MANUAL, 2d ed., 1989, at pages 14.18-14.19: A first cycle, comprising (i) a denaturation step for 5 minutes at 94° C., (ii) an annealing step for 2 min at 50° C., (iii) a polymerisation step for 3 min at 72° C., subsequent cycles, comprising (i) a denaturation step for 1 minute at 94° C., (ii) an annealing step for 2 min at 50° C., (iii) a polymerisation step for 3 min at 72° C., and a last cycle, comprising (i) a denaturation step for 5 minutes at 94° C., (ii) an annealing step for 2 min at 50° C., (iii) a polymerisation step for 10 min at 72° C., all steps being performed in the appropriate buffer solutions as proposed by the authors.

“Random sequences”, within the meaning of the invention, are nucleotide sequences being determined by a stochastic process, or the sequences which are created by combinatorial processes, e.g., by a computer program, and which need not necessarily have a biological meaning.

An “oligonucleotide array”, within the meaning of the invention, shall be understood as a device on the planar surface of which there are nucleic acid molecules immobilised and for which the sequence of the nucleic acid molecules that are immobilised at a certain part of the planar surface of the device is known.

A “pattern of detected hybridisation complexes”, within the meaning of the invention, is to be understood as the combination of signals that are obtained by hybridisation and detection of immune genes. The “pattern of detected hybridisation complexes” represents the information content that is obtained from a hybridisation and detection experiment. These patterns can be compared manually by a skilled individual or automatically by, e.g., a computer program. Computer programs and algorithms for pattern recognition are well known to the skilled artisan. Computer programs suitable for pattern recognition or pattern comparison within the meaning of the invention apply, e.g., support vector machines, fuzzy logic algorithms, artificial neural networks, principle component analysis, expert systems, clustering algorithms, and/or other pattern recognition algorithms. Comparison of patterns, within the meaning of the invention, can be with the patterns obtained in contemporaneous control experiments or with patterns from previous experiments, from data reported in literature, or other sources.

It is an object of the invention to provide a method to characterise the immune gene repertoire of a vertebrate comprising the steps of (i) collecting a sample comprising suitable cells from the vertebrate, (ii) preparing from said sample nucleic acid molecules representing the immune gene repertoire, (iii) hybridizing the nucleic acid molecules of (ii) to immobilised oligonucleotides, thereby forming hybridisation complexes; and detecting said hybridisation complexes.

It is another object of the invention to provide a method for detecting the presence of a specific immune gene in a vertebrate comprising the steps of (i) collecting a sample comprising suitable cells from the vertebrate, (ii) preparing from said sample nucleic acid molecules representing the immune gene repertoire, (iii) hybridizing the nucleic acid molecules of (ii) to immobilised oligonucleotides, thereby forming a hybridisation complexes; and detecting said hybridisation complexes.

It is another object of the invention to provide the above method to characterise the immune gene repertoire of a vertebrate or the above method to detect the presence of a specific immune gene in a vertebrate in which at least part of the variable region of the immune gene or immune genes is/are amplified prior to hybridisation.

It is another object of the invention to provide one of the above methods in which the part of the variable region to be amplified is a CDR3 region of a TCR and/or a CDR3 region of an immunoglobulin heavy chain and/or light chain.

It is another object of the invention to provide one of the above methods in which the variable region to be amplified is a CDR2 or CDR1 region.

It is ther object of the invention to provide one of the above methods in which PCR or in vitro transcription is used for the amplification step.

Yet another object of the invention are methods of the above wherein the variable region of the immune gene is amplified using a 5′ primer selected from a group of primers consisting of SEQ ID NO:5, SEQ ID NO:6, and SEQ ID NO:7 or a 5′ primer comprising the consensus sequence depicted in SEQ ID NO:1, and a 3′ primer having a sequence which hybridises under conditions compatible with PCR to nucleic acid molecules having the sequence of SEQ ID NO:2 or SEQ ID NO:3. 5′ Primers comprising the sequence of SEQ ID NO:1 are, e.g., well suited to amplify the CDR3 region of various TCR-beta families.

Immobilisation of the nucleic acid molecules, according to the invention, can be on glass, silicon, nitrocellulose, or on other solid surface materials.

Preferred cells collected in a method according to the invention can be, e.g., blood cells, B lymphocytes and/or T lymphocytes. Preferred nucleic acid molecules obtained in step (ii) of the above methods are nucleic acid molecules that represent the variable regions of B-cell receptors and/or T-cell receptors.

Methods of the invention can use immobilised nucleic acid molecules with random sequences in step (iii) of the above methods. These random sequences are preferably 7 to 15, more preferred 8-10, most preferred 9 nucleotides in length.

It is another object of the invention to provide the above methods to characterise the immune gene repertoire of a vertebrate or the above method to detect the presence of a specific immune gene in a vertebrate in which the immobilised sequences are known to be comprised in nucleic acid molecules that code for the variable region of antibodies or T-cells, or complementary sequences.

Nucleic acid molecules immobilised in methods of the invention can be, e.g., RNA or DNA.

Methods of the invention encompass methods in which the nucleic acid molecules are immobilised on a solid support, preferably on an oligonucleotide array. The immobilised nucleic acid molecules can also be immobilised on nitrocellulose or on a paper support.

Methods of the invention encompass methods in which the nucleic acid molecules are labeled. It is a preferred embodiment of the invention that the label is a fluorescent label or the label is a radioactive label, or the label is a luminescent label.

Methods of the invention can be applied to humans.

It is another object of the invention to provide a kit containing the material necessary to perform the methods of the invention as described above. A kit according to the invention may comprise, e.g., a set of primers, an oligonucleotide array, suitable buffer solutions and/or other reagents nescessary to perform methods of the invention.

Another object of the invention is a method of identifying an immune disorder in a vertebrate from a sample comprising suitable cells of said vertebrate comprising the steps of (i) preparing nucleic acid molecules representing the immune gene repertoire of the vertebrate to be tested from said sample, (ii) incubating the nucleic acid molecules of (i) to immobilised oligonucleotides, thereby forming hybridisation complexes, (iii) detecting said hybridisation complexes; and (iv) comparing the pattern of detected hybridisation complexes with the pattern of detected hybridisation complexes of healthy and/or diseased vertebrates. An immune disorder can then be diagnosed, e.g., if the pattern of detected hybridisation complexes of the vertebrate tested resembles the pattern of detected hybridisation complexes of a diseased vertebrate.

Another object of the invention is a method for identifying compounds that increase or reduce the transcription of an immune gene, the number of immune receptors and/or the number of immune cells in a vertebrate comprising the steps of (i) collecting a sample comprising suitable cells from a vertebrate, (ii) preparing from said sample nucleic acid molecules representing the immune gene repertoire, (iii) hybridizing the nucleic acid molecules of (ii) to immobilised oligonucleotides, thereby forming hybridisation complexes, (iv) detecting said hybridisation complexes, (v) comparing the pattern of detected hybridisation obtained in the presence of the compound with the pattern of detected hybridisation complexes obtained in the absence of the compound. A compound can be identified as increasing or reducing the transcription of said immune gene, or increasing or reducing the production of the immune receptor and/or the immune cell if it can be seen from the pattern of detected hybridisation complexes that the transcription or production of said immune gene, immune receptor and/or immune cell is increased or reduced.

Use of a compound identified with a method of the above for the treatment of an immune disease.

A method for the preparation of a pharmaceutical composition for treating an immune disorder in a vertebrate comprising the steps of (i) collecting samples comprising suitable cells from diseased and healthy vertebrates, (ii) preparing from said samples nucleic acid molecules representing the immune gene repertoires of the diseased and healthy vertebrates, (iii) hybridizing the nucleic acid molecules of (ii) to immobilised oligonucleotides, thereby forming hybridisation complexes, (iv) detecting said hybridisation complexes, (v) comparing the pattern of detected hybridisation complexes of the healthy and the diseased vertebrates, and (vi) preparing a pharmaceutical composition comprising at least one immune gene, immune receptor and/or immune cell, which is in higher or lower abundance in a diseased vertebrate as compared to a healthy vertebrate.

A method for the preparation of a pharmaceutical composition for treating an immune disorder in a vertebrate comprising the steps of (i) collecting samples comprising suitable cells from diseased and healthy vertebrates, (ii) preparing from said samples nucleic acid molecules representing the immune gene repertoires of the diseased and healthy vertebrates, (iii) hybridizing the nucleic acid molecules of (ii) to immobilised oligonucleotides, thereby forming hybridisation complexes, (iv) detecting said hybridisation complexes, (v) preparing a pharmaceutical composition comprising at least one agent that stimulates or reduces the production of an immune gene, immune receptor and/or immune cell, which is in lower or higher abundance in a diseased vertebrate as compared to a healthy vertebrate.

The invention further comprises a pharmaceutical composition obtained by the above mentioned methods.

The invention further comprises methods of the above in which support vector machines are used.

The invention further comprises methods of the above in which fuzzy logic, artificial neural networks, principle component analysis, expert systems, or clustering algorithms are used.

The invention will be further described in the following examples, which do not limit the scope of the invention described in the claims. The following examples are offered for illustrative purposes only, and are not intended to limit the scope of the present invention in any way.

### EXAMPLES

All commercially available reagents referred to in the examples were used according to manufacturer's instructions unless otherwise indicated.

### Example 1

### VH-Gene Expression Analysis

Peripheral blood mononuclear cells (PBMC) were obtained by density sedimentation (LSM, Organon Teknika, Durham, N.C.) from 10 ml of whole blood. Isolation of RNA was done according to the procedure recommended by Affymetrix in the GeneChip Expression Analysis technical manual. In brief, PMBC were lysed in TRIzol reagent and total RNA was isolated using QIAGEN's RNeasy total RNA isolation kit. Double-stranded cDNA was prepared using the Invitrogen Life Technologies SuperScript Choice system. Instead of the oligo (dT), T7-(dT)24 oligomer or random primers the primer T7(CH1) was used for priming first-strand cDNA synthesis.

(SEQ ID NO:4)

Primer T7(CH1):
5′-GGCCAGTGAATTGTAATACGACTCACTATAGGGAGGCGGGAAACACG
CTTGGACCTTTGGTCGACGCTGAGCTAACCGT-3′

Primer hybridisation to RNA was done in an RNAse free Eppendorf tube at 70° C. for 10 minutes in DEPC-H₂O. Subsequently, the reaction was spun down and put on ice. 5× first strand cDNA buffer, and 0.1 mM dNTP mix was added to the tube, mixed and incubated at 42° C. for 2 minutes for temperature adjustment. SuperScript II RT was added and the whole reaction mixture was incubated for 1 hour. For second strand cDNA synthesis the reaction was put on ice, DEPEC-treated water, 5× second strand reaction buffer, 10 mM DNTP mix, 10 U/μl E. coli DNA Ligase, 10 U/μl E. coli DNA Polymerase I, and 2 U/μl E. coli RNase H was added, mixed and incubated at 16° C. for 2 hours in a cooling waterbath. Subsequently, 10 U T4 DNA polymerase were added and the mixture was incubated for another 5 minutes at 16° C., before the reaction was stopped by addition of 0.5 M EDTA. Double stranded cDNA was purified using Phase Lock Gels-phenol/chloroform extraction. Biotin labeled cRNA was synthesised using the BioArray HighYield RNA transcript labeling kit of ENZO. In vitro transcription products were purified using RNeasy spin columns from QIAGEN and the resulting cRNA was fragmented at a concentration of 0.5 μg/ml in fragmentation buffer at 94° C. for 35 minutes. Hybridisation of fragmented cRNA to GeneChip® arrays of Affymetrix Inc. was done as described in the GeneChip® Expression Analysis technical manual. In brief, a hybridisation cocktail was prepared with 10 μg fragmented cRNA, 3.3 μL control oligonucleotide B2 (3 nM), 10 μL 20× Eukaryotic hybridisation controls (bioB, bioC, bioD, cre), 2 μL herring sperm DNA (10 mg/mL), 2 μL acetylated BSA (50 mg/mL), 100 μL 2× hybridisation buffer (Final 1× concentration is 100 mM MES, 1M [Na⁺], 20 mMEDTA, 0.01% Tween 20), filled to a final volume of 200 μL with H₂O. The oligonucleotide array was equilibrated to room temperature immediately before use, filled with 1× hybridisation buffer and incubated at 45° C. for 10 min with rotation. The hybridisation cocktail was incubated at 99° C. for 5 min and subsequently at 45° C. for 5 min. The hybridisation cocktail was spun down at maximum speed in a microcentrifuge for 5 min to remove any insoluble material from the hybridisation mixture. Then, the buffer solution from the probe array cartridge was removed and the array was filled with appropriate volume of clarified hybridisation cocktail. The array was placed in a rotisserie box in a 45° C. oven and hybridisation of the labelled nucleic acids to the array was allowed for 16 hours. Washing, staining and scanning was done using the Affymetrix GeneChip® instrument system, consisting of a workstation with the software program Affymetrix® Microarray Suite, fluidic station 400, and Genearray scanner™. The antibody signal amplification protocol for eukaryotic targets was used for washing and staining with streptavidin phycoerythrin as described in the GeneChip® Expression Analysis technical manual. Scanning was done at 570 nm. The Micro-array Suite generated a dat.file and cel.file.

### Example 2

### T Cell Receptor Beta Gene Expression Analysis

Peripheral blood mononuclear cells (PBMC) were obtained by density sedimentation (LSM, Organon Teknika, Durham, N.C.) from 10 ml of whole blood. Isolation of RNA was done according to the procedure recommended by Affymetrix in the GeneChip Expression Analysis technical manual. In brief, PMBC were lysed in TRIzol reagent and total RNA was isolated using QIAGEN's RNeasy total RNA isolation kit. First strand cDNA was prepared using the Invitrogen Life Technologies SuperScript First Strand Synthesis System with the oligo(dT) oligomer for priming first-strand cDNA synthesis. RNA/Primer mixtures were prepared in sterile 0.5 ml tubes using up to 5 μg total RNA, 1 μl of 10 mM dNTP mix, 1 μl of 0.5 μg/μl Oligo(dT), filled to 10 μl with DEPEC treated H₂O. The samples were incubated at 65° C. for 5 min, then placed on ice for at least 1 min. For each sample an reaction mixture was prepared, adding each component in the following order. 2 μl 10X RT buffer, 4 μl of 25 mM MgCl₂, 2 μl of 0.1 M DTT and 1 μl of RNaseOUT Recombinant RNase Inhibitor. 9 μl of the reaction mixture were added to each RNA/primer mixture, mixed, collected by brief centrifugation, and incubated at 42° C. for 2 min. Then, 1 μl (50 units) of SUPERSCRIPT II RT was added to each tube, mixed, and incubated at 42° C. for 50 min. The reaction was terminated at 70° C. for 15 min and subsequently put on ice. The reaction was collected by brief centrifugation, 1 μl of RNase H was added to each tube and incubated for 20 min at 37° C.

An aliquot of the cDNA synthesis reaction (corresponding to 200 ng of total RNA) was amplified in a 50 μl multiplex reaction with V beta oligonucleotides 1, 2, 3, and the T7-C-beta oligonucleotide on a Biometra PCR system (Biometra, Göttingen, Germany).

(SEQ ID NO:5)

Oligo V beta1:
TATTTCTGTGCCAGCAG

(SEQ ID NO:6)

Oligo V beta2:
TGTATCTCTGTGCCAGCAG

(SEQ ID NO:7)

Oligo V beta3:
TGTACTTCTGTGCCAGCAG

(SEQ ID NO:8)

Oligo T7-C-beta:
GGCCAGTGAATTGTAATACGACTCACTATAGGGAGGCGGAAACACAGCGA
CCTCGGGTGGGAACAC

The reaction contained 500 μM dNTPs, 2.0 mM MgCl₂, 1 unit AmpliTaq Gold DNA polymerase (Perkin Elmer) for hot start in 1× buffer. The final concentration of each primer was 0.5 μM. The PCR conditions were: an initial incubation of 95° C. for 7 min, followed by 25-35 cycles of 94° C. for 30 s, 58° C. for 30 s, 72° C. for 30 s, and finally one incubation step of 72° C. for 10 min. PCR products were purified using the QIAquick PCR purification kit (Qiagen, Hilden, Germany).

Biotin labeled cRNA was synthesised from the PCR reaction product using the BioArray HighYield RNA transcript labeling kit of ENZO. In vitro transcription products were purified using RNeasy spin columns from QIAGEN and the resulting cRNA was fragmented at a concentration of 0.5 μg/ml in fragmentation buffer at 94° C. for 35 minutes. Hybridisation of fragmented cRNA to GeneChip® arrays of Affymetrix Inc. was done as described in the GeneChip® Expression Analysis technical manual. In brief, a hybridisation cocktail was prepared with 10 μg fragmented cRNA, 3.3 μL control oligonucleotide B2 (3 nM), 10 μL 20× Eukaryotic hybridisation controls (bioB, bioC, bioD, cre), 2 μL herring sperm DNA (10 mg/mL), 2 μL acetylated BSA (50 mg/mL), 100 μL 2× hybridisation buffer (Final 1× concentration is 100 mM MES, 1M [Na⁺], 20 mMEDTA, 0.01% Tween 20), filled to a final volume of 200 μL with H₂O. The oligonucleotide array was equilibrated to room temperature immediately before use, filled with 1× hybridisation buffer and incubated at 45° C. for 10 min with rotation. The hybridisation cocktail was incubated at 99° C. for 5 min and subsequently at 45° C. for 5 min. The hybridisation cocktail was spun down at maximum speed in a microcentrifuge for 5 min to remove any insoluble material from the hybridisation mixture. Then, the buffer solution from the probe array cartridge was removed and the array was filled with appropriate volume of clarified hybridisation cocktail. The array was placed in a rotisserie box in a 45° C. oven and hybridisation of the labelled nucleic acids to the array was allowed for 16 hours. Washing, staining and scanning was done using the Affymetrix GeneChip® instrument system, consisting of a workstation with the software program Affymetrix® Microarray Suite, fluidic station 400, and Genearray scanner™. The antibody signal amplification protocol for eukaryotic targets was used for washing and staining with streptavidin phycoerythrin as described in the GeneChip® Expression Analysis technical manual. Scanning was done at 570 μm. The Micro-array Suite generated a dat.file and cel.file.

### Example 3

### Analysis of Immune Gene Hybridisation Pattern Using Support Vector Machines

Support vector machines (SVM) are well suited for two-class or multi-class pattern recognition (Weston and Watkins, Proceedings of the Seventh European Symposium On Artificial Neural Networks, April 1999; Vapnik, The Nature of Statistical Learning Theory, 1995, Springer, New York, Vapnik, Statistical Learning Theory, 1998, Wiley, New York; Burges, Data Mining and Knowledge Discovery, 2(2): 955-974, 1998). For the two-class classification problem, assume that we have a set of samples, i.e., a series of input vectors \vec{x}ᵢ∈R^{d} (i=1, 2, . . . , m) with corresponding labels yᵢ∈{+1,−1} (i=1, 2, . . . , m). Here, +1 and −1 indicate the two classes. To classify gene expression patterns of rearranged immune genes for describing the current immune status, the input vector dimension is equal to the number of different oligonucleotide types present on the oligonucleotide array or a subset hereof, and each input vector unit stands for the hybridisation value of one specific oligo-nucleotide type. The goal is to construct a binary classifier or derive a decision function from the available samples which has a small probability of misclassifying a future sample.

An SVM implements the following idea: it maps the input vectors \vec{x}ᵢ∈R^{d} into a high-dimensional feature space Φ(\vec{x})∈H and constructs an Optimal Separating Hyperplane (OSH), which maximises the margin, the distance between the hyperplane and the nearest data points of each class in the space H (see FIG. 1). By choosing bSH from among the many that can separate the positive from the negative examples in the feature space, SVMs are avoiding the risk of overfitting.

Different mappings construct different SVMs. The mapping Φ: R^{d} [symbol] H is performed by a kernel function K(\vec{x}ᵢ,\vec{x}ⱼ) which defines an inner product in the space H.

The decision function implemented by SVM can be written as (Burges, Data Mining and Knowledge Discovery, 2(2): 955-974, 1998):

$$f ( \vec{x} ) = sgn ( {\sum }_{i = 1}^{n} {y}_{i} {α}_{i} \cdot K ( \vec{x} , {\vec{x}}_{i} ) + b ) \qquad ( 1 )$$

where the coefficients αᵢ are obtained by solving the following convex Quadratic Programming (QP) problem:

$$Maximise {\sum }_{i = 1}^{m} {α}_{i} - \frac{1}{2} {\sum }_{i = 1}^{m} {\sum }_{j = 1}^{m} {α}_{i} {α}_{j} \cdot {y}_{i} {y}_{j} \cdot K ( {\vec{x}}_{i} , {\vec{x}}_{j} ) \\ subject to 0 \leq {α}_{i} \leq C and {\sum }_{i = 1}^{m} {α}_{i} {y}_{i} = 0 \qquad ( 2 )$$

The regularity parameter C (equation 2) controls the trade off between margin and misclassification error. The \vec{x}ⱼ are called Support Vectors only if the corresponding αᵢ>0.

Two of the kernel functions used in the current example:

$$\begin{matrix}K ( {\vec{x}}_{i} , {\vec{x}}_{j} ) = {( {\vec{x}}_{i} \cdot {\vec{x}}_{j} + 1 )}^{d} & ( 3 ) \\ K ( {\vec{x}}_{i} , {\vec{x}}_{j} ) = {ⅇ}^{( - r {‖ {\vec{x}}_{i} , {\vec{x}}_{j} ‖}^{2} )} & ( 4 )\end{matrix}$$

where the first one (equation 3) is called the polynomial kernel function of degree d which will eventually revert to the linear function when d=1, the latter (equation 4) is called the Radial Basic Function (RBF) kernel.

For a given data set, only the kernel function and the regularity parameter C must be selected to specify one SVM. An SVM has many attractive features. For instance, the solution of the QP problem is globally optimised while with neural networks the gradient based training algorithms only guarantee finding a local minima. In addition, SVM can handle large feature spaces, can effectively avoid overfitting (see above) by controlling the margin, can automatically identify a small subset made up of informative points, i.e., the Support Vectors, etc.

The classification of current immune status of a vertebrate and thereby the identification of an disorder based on gene expression data is a multi-class classification problem. The class number k is equal to the number of immune states/disorders which should be predicted, i.e., which are present in the training data set. Due to the limited number of different classes in the present sample set, we decided to handle the multi-class classification by reducing the multi-classification to a series of binary classifications. For a k-class classification, k SVMs are constructed. The ith SVM will be trained with all of the samples in the ith class with positive labels and all other samples with negative labels. Finally an unknown sample is classified into the class that corresponds to the SVM with the highest output value. This method is used to construct a prediction/classification system for gene expression patterns of rearranged immune genes.

Each data point generated by a microarray hybridisation experiment (cf. example 1 and 2) corresponds to and is determined by the number of mRNA copies present in the analysed sample, i.e., from an experiment with n oligonucleotide types on a polynucleotide array, a series of n expression-level values is obtained. These n values are typically stored in a metrics file which is the result of the analysis of a “cel file” by the Affymetrix® Microarray Suite. The data from a series of m metrics files (representing m hybridisation experiments) are taken to build an expression matrix, in which each of the m rows consists of an n-element expression vector for a single experiment. In order to normalise the expression values of the m experiments, we define x_{i,j} to be the sum of the logarithms of the expression level a_{i,j} for gene j (whose mRNA hybridises with the oligonucleotide type j′ present on the microarray), normalised so that the expression vector \vec{x}ᵢ has the Euclidean length l:

$${x}_{j , i} = \frac{ln ( {a}_{i , j} )}{\sqrt{{\sum }_{k = 1}^{n} {ln ( {a}_{i , k} )}^{2}}} \qquad ( 5 )$$

Initial analyses are carried out using a set of 20000-element expression vectors for 297 experiments as described in example 1 and 2 (240 experiments in the training set and 57 in the test set).

Using the knowledge that the 297 experiments represent three different immune states, we trained the SVMs described above with the training set to recognise those immune states. The test set was used to assess the prediction accuracy.

1. A method to characterize the immune gene repertoire of a vertebrate comprising the steps of
i) collecting a sample comprising suitable cells from the vertebrate,
ii) preparing from said sample nucleic acid molecules representing the immune gene repertoire,
iii) hybridizing the nucleic acid molecules of (ii) to immobilised oligonucleotides, thereby forming hybridization complexes; and
iv) detecting said hybridization complexes.

2. A method to detect the presence of a specific immune gene in a vertebrate comprising the steps of
i) collecting a sample comprising suitable cells from the vertebrate,
ii) preparing from said sample nucleic acid molecules representing the immune gene repertoire,
iii) hybridizing the nucleic acid molecules of (ii) to immobilized oligonucleotides, thereby forming a hybridization complexes; and
iv) detecting said hybridization complexes.

3. Method of claim 1 wherein the preparation step in (ii) comprises amplification of the variable region of the immune gene or genes.

4. Method of claim 3 wherein the variable region to be amplified is a CDR3 region.

5. Method of claim 3 wherein the variable region to be amplified is the CDR3 5 region of the heavy chain.

6. Method of claim 3 wherein the variable region to be amplified is a CDR2 or CDR1 region.

7. Method of claim 3 wherein the amplification step is by PCR or by in vitro transcription.

8. Method of claim 3 wherein the variable region of the immune gene is amplified using a 5′ primer selected from a group of primers consisting of SEQ ID NO:5, SEQ ID NO:6, and SEQ ID NO:7 or a 5′ primer comprising the consensus sequence depicted in SEQ ID NO:1, and a 3′ primer having a sequence which hybridises under conditions compatible with PCR to nucleic acid molecules having the sequence of SEQ ID NO:2 or SEQ ID NO:3.

9. Method of claim 1 or 2 wherein the oligonucleotide of (iii) is immobilised on glass, silicon, or nitrocellulose.

10. Method of claim 1 or 2 wherein the cells in (i) are blood cells.

11. Method of claim 1 or 2 wherein the cells in (i) are B lymphocytes and/or T lymphocytes.

12. Method of claim 1 or 2 wherein the nucleic acid molecules of (ii) represent the variable regions of the B-cell receptors and/or T-cell receptors.

13. Method of claim 1 wherein the immobilised nucleic acid molecules in (iii) are nucleic acid molecules with random sequences.

14. Method of claim 13 wherein the random sequences are 7 to 15 nucleotides in length.

15. Method of claim 1 or 2 wherein the immobilised nucleic acid molecules are sequences known to be comprised in nucleic acid molecules that code for the variable region of antibodies or T-cell receptors, or complementary sequences.

16. Method of claim 1 or 2 wherein the immobilised nucleic acid molecules are DNA.

17. Method of claim 1 or 2 wherein the immobilised nucleic acid molecules are RNA.

18. Method of claim 1 or 2 wherein the immobilised nucleic acid molecules are immobilised on a solid support.

19. Method of claim 1 or 2 wherein the immobilised nucleic acid molecules are immobilised on an oligonucleotide array.

20. Method of claim 1 or 2 wherein the immobilised nucleic acid molecules are immobilised on a nitrocellulose or paper support.

21. Method of claim 1 or 2 wherein the nucleic acid molecules are labeled.

22. Method of claim 21 wherein the label is fluorescent, luminescent or radioactive.

23. Method of claim 1 or 2 wherein the vertebrate is a human.

24. A diagnostic kit containing the material necessary to perform any of the methods of claim 1 to 23.

25. A method of identifying an immune disorder in a vertebrate from a sample comprising suitable cells of said vertebrate comprising the steps of
i) preparing nucleic acid molecules representing the immune gene repertoire of the vertebrate to be tested from said sample,
ii) incubating the nucleic acid molecules of (i) to immobilised oligonucleotides, thereby forming hybridization complexes,
iii) detecting said hybridization complexes; and
iv) comparing the pattern of detected hybridization complexes with the pattern of detected hybridization complexes of healthy and/or diseased vertebrates.

26. A method for identifying compounds that increase or reduce the transcription of at least one immune gene, the number of immune receptors and/or the number of immune cells in a vertebrate comprising the steps of
i) collecting a sample comprising suitable cells from a vertebrate,
ii) preparing from said sample nucleic acid molecules representing the immune gene repertoire,
iii) hybridizing the nucleic acid molecules of (ii) to immobilised oligonucleotides, thereby forming hybridization complexes,
iv) detecting said hybridization complexes, and
v) comparing the pattern of detected hybridization complexes obtained in the presence of the compound with the pattern of detected hybridisation complexes obtained in the absence of the compound.

27. A method for the treatment of an immune disorder comprising administering to a vertebrate an effective amount of a compound identified by the method of claim 26.

28. A method for the preparation of a pharmaceutical composition for treating an immune disorder in a vertebrate comprising the steps of
i) collecting samples comprising suitable cells from diseased and healthy vertebrates,
ii) preparing from said samples nucleic acid molecules representing the immune gene repertoires of the diseased and healthy vertebrates,
iii) hybridizing the nucleic acid molecules of (ii) to immobilized oligonucleotides, thereby forming hybridization complexes,
iv) detecting said hybridization complexes,
v) comparing the pattern of detected hybridization complexes of the healthy and the diseased vertebrates, and
vi) preparing a pharmaceutical composition comprising at least one immune gene, immune receptor and/or immune cell, which is in higher or lower abundance in a diseased vertebrate as compared to a healthy vertebrate.

29. A method for the preparation of a pharmaceutical composition for treating an immune disorder in a vertebrate comprising the steps of
i) collecting samples comprising suitable cells from diseased and healthy vertebrates,
ii) preparing from said samples nucleic acid molecules representing the immune gene repertoires of the diseased and healthy vertebrates,
iii) hybridizing the nucleic acid molecules of (ii) to immobilised oligonucleotides, thereby forming hybridisation complexes,
iv) detecting said hybridization complexes, and
v) preparing a pharmaceutical composition comprising at least one agent that stimulates or reduces the production of an immune gene, immune receptor and/or immune cell, which is in lower or higher abundance in a diseased vertebrate as compared to a healthy vertebrate.

30. A pharmaceutical composition obtained by the method of claim 28 or 29.

31. A method of any of claims 1 to 23, or claim 25, or claim 26, or claim 28, or claim 29, wherein support vector machines are used.

32. A method of any of claims 1 to 23, or claim 25, or claim 26, or claim 28, or claim 29, wherein fuzzy logic, artificial neural networks, principle component analysis, expert systems, or clustering algorithms are used.

33. Method of claim 2 wherein the preparation step in (ii) comprises amplification of the variable region of the immune gene or genes.

34. Method of claim 33 wherein the variable region to be amplified is a CDR3 region.

35. Method of claim 33 wherein the variable region to be amplified is the CDR3 5 region of the heavy chain.

36. Method of claim 33 wherein the variable region to be amplified is a CDR2 or CDR1 region.

37. Method of claim 33 wherein the amplification step is by PCR or by in vitro transcription.

38. Method of claim 33 wherein the variable region of the immune gene is amplified using a 5′ primer selected from a group of primers consisting of SEQ ID NO:5, SEQ ID NO:6, and SEQ ID NO:7 or a 5′ primer comprising the consensus sequence depicted in SEQ ID NO:1, and a 3′ primer having a sequence which hybridises under conditions compatible with PCR to nucleic acid molecules having the sequence of SEQ ID NO:2 or SEQ ID NO:3.

39. Method of claim 2 wherein the immobilised nucleic acid molecules in (iii) are nucleic acid molecules with random sequences.

40. Method of claim 39 wherein the random sequences are 7 to 15 nucleotides in length.


SEQUENCE LISTING (8 sequences)

SEQ ID NO: 1 - DNA, 9 bp, Artificial
  TCRB 3′ Primer Consensus Sequence
tgtgccagc

SEQ ID NO: 2 - DNA, 534 bp, Homo sapiens
gaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 3 - DNA, 540 bp, Homo sapiens
gaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 4 - DNA, 79 bp, Artificial
  Primer T7(CH1)
ggccagtgaattgtaatacgactcactatagggaggcgggaaacacgcttggacctttggtcgacgctgagctaaccgt

SEQ ID NO: 5 - DNA, 17 bp, Artificial
  Oligo V beta1
tatttctgtgccagcag

SEQ ID NO: 6 - DNA, 19 bp, Artificial
  Oligo V beta2
tgtatctctgtgccagcag

SEQ ID NO: 7 - DNA, 19 bp, Artificial
  Oligo V beta3
tgtacttctgtgccagcag

SEQ ID NO: 8 - DNA, 66 bp, Artificial
  Oligo T7-C-beta
ggccagtgaattgtaatacgactcactatagggaggcggaaacacagcgacctcgggtgggaacac